(19) United States
(12) Patent Application Publication
(10) Pub. No.: US 2013/0103015 A1
(43) Pub. Date: Apr. 25, 2013

(54) OCT-Guided Femtosecond Laser to Measure a Retinal Surface for Use in Performing an Intra-Retinal Ablation

(71) Applicants: Robert Edward Grant, Laguna Beach, CA (US); David Haydn Mordaunt, Los Gatos, CA (US)

(72) Inventors: Robert Edward Grant, Laguna Beach, CA (US); David Haydn Mordaunt, Los Gatos, CA (US)

(21) Appl. No.: 13/405,097

(22) Filed: Feb. 24, 2012

Related U.S. Application Data
(60) Provisional application No. 61/549,932, filed on Oct. 21, 2011.

(51) Int. Cl. A61F 9/011

(52) U.S. Cl. 606/6

(57) ABSTRACT

A system and method are provided for debulking scar tissue in the retina by the Laser Induced Optical Breakdown (LIOB) of scar tissue. The identification, location and extent of the scar tissue is accomplished using Optical Coherence Tomography (OCT) techniques. OCT imaging is also used to monitor the debulking procedure.

This application claims the benefit of U.S. Provisional Patent Application Ser. No. 61/549,932, filed Oct. 21, 2011.

### FIELD OF THE INVENTION

The present invention pertains generally to systems and methods for performing ophthalmic laser surgical procedures. More particularly, the present invention pertains to systems and methods for performing an ablation of ophthalmic tissue for the purpose of debulking scar tissue in the retina. The present invention is particularly, but not exclusively, useful as a laser surgical procedure wherein scar tissue in the retina is located and identified using Optical Coherence Tomography (OCT) techniques, and the scar tissue is then debulked by causing Laser Induced Optical Breakdown (LIOB) of the scar tissue.

### BACKGROUND OF THE INVENTION

It sometimes happens that therapeutic injections of biologics or pharmacologics into the retina of an eye can cause a reaction from retinal tissue that is known as tachyphylaxis. The undesirable consequence here is the formation of retinal scar tissue. Typically, this scar tissue develops inside the retina rather than on the surface of the retina. Nevertheless, even though the retinal surface may remain functional, tachyphylaxis can eventually cause a considerable diminution in visual acuity. In the first instance, tachyphylaxis is obviously to be avoided. If it occurs, however, it becomes necessary for the scar tissue to be somehow removed.

As implied above, when scar tissue is to be removed from the retina of an eye, it is most desirable (i.e. crucial) that the functionality of the retinal surface be preserved to the greatest extent possible. To do this, it is necessary that the retinal surface not be unduly disturbed. This, in turn, requires a removal of scar tissue from the retina that is accomplished with extreme precision and effectiveness. In the event, the removal of scar tissue from within the retina can require operational tolerances as small as 10-50 microns. With this in mind, it is known that femtosecond laser systems can be operated to perform tissue ablation by Laser Induced Optical Breakdown (LIOB) within such tolerances. Also, it is known that Optical Coherence Tomography (OCT) imaging units can create images that distinguish structures within such tolerances.

In light of the above, it is an object of the present invention to provide a system and method for using OCT imaging techniques for the purpose of identifying the location and extent of scar tissue in the retina of an eye. Another object of the present invention is to provide a system and method for using OCT images as information for guiding a laser unit to perform LIOB on scar tissue inside the retina of an eye. Yet another object of the present invention is to provide a system and method that uses OCT imaging techniques to guide a laser beam, during the intra-retinal ablation of tissue by LIOB, for debulking scar tissue that has formed in the retina. Still another object of the present invention is to provide an ophthalmic system and its method of use for debulking scar tissue in a retina that is easy to use, is simple to manufacture, and is comparatively cost effective.

### SUMMARY OF THE INVENTION

In accordance with the present invention, a laser beam is used for the purpose of debulking scar tissue that has formed in the retina of an eye as a result of tachyphylaxis. Specifically, the scar tissue is debulked by performing Laser Induced Optical Breakdown (LIOB) on the tissue. To do this, an imaging unit is used to first identify, locate and measure the extent of the scar tissue. The imaging unit is then used to monitor the debulking procedure, and provide input to a computer/comparator for control of the laser beam during the debulking procedure.

Structurally, the system for performing an intra-retinal ablation of scar tissue in accordance with the present invention includes a laser unit for generating a laser beam. Importantly, the laser beam must be capable of performing LIOB on scar tissue in the retina. Preferably, the laser beam is a pulsed femtosecond laser beam, and the laser unit includes optics for focusing the laser beam to an approximately ten micron diameter focal point. Further, the system includes a computer for guiding the focal point of the laser beam along a predetermined path in the scar tissue to debulk the scar tissue by LIOB.

For purposes of the present invention, the imaging unit is provided to create a three dimensional image of a region of the retina of an eye. More specifically, the three dimensional image that is created includes information about the thickness “t” of the retina. It also includes information about variations “Δt” in the thickness of the retina in the region being imaged that are indicative of scar tissue in the retina. Preferably, the imaging unit is a type that employs OCT imaging techniques.

In addition to the laser unit and the imaging unit, the system of the present invention also includes an analyzer for evaluating the three dimensional retinal image that is created by the imaging unit. As indicated above, this evaluation is based on variations of retinal thickness “Δt”, and is done to identify a location and an extent of the scar tissue inside the retina. This information is then provided as input to the computer.

Based on the identification and location of scar tissue in the image that is created by the imaging unit, the computer/comparator provides control for the laser unit. Specifically, this control is provided to guide the focal point of the laser beam along a path that is maintained beyond a predetermined distance “d”, behind the surface of the retina. Typically, the distance “d” is greater than approximately ten microns. To ensure this happens, the comparator portion of the computer/comparator uses input from the imaging unit to monitor the movement of the laser beam focal point, in real time. The purpose here is essentially two-fold. For one, this is done to measure a deviation of the focal point from the predetermined path that is presented in the three dimensional image created by the imaging unit. Control of the laser unit is then provided to minimize any deviation of the focal point from the predetermined path. For another, control of the focal point is accomplished to minimize “Δt”, to thereby debulk scar tissue inside the retina.

### BRIEF DESCRIPTION OF THE DRAWINGS

The novel features of this invention, as well as the invention itself, both as to its structure and its operation, will be best understood from the accompanying, drawings, taken in conjunction with the accompanying description, in which similar reference characters refer to similar parts, and in which:

FIG. 1 is a schematic presentation of components for an ophthalmic system, shown in an operational relationship with an eye (shown in cross-section), for debulking scar tissue in the eye by intra-retinal ablation;

FIG. 2 is a cross-section view of the posterior of the eye, as seen along the line 2-2 in FIG. 1, showing the presence of scar tissue in the retina; and

FIG. 3 is a view of the retina as seen in FIG. 2 after removal of the scar tissue.

### DESCRIPTION OF THE PREFERRED EMBODIMENTS

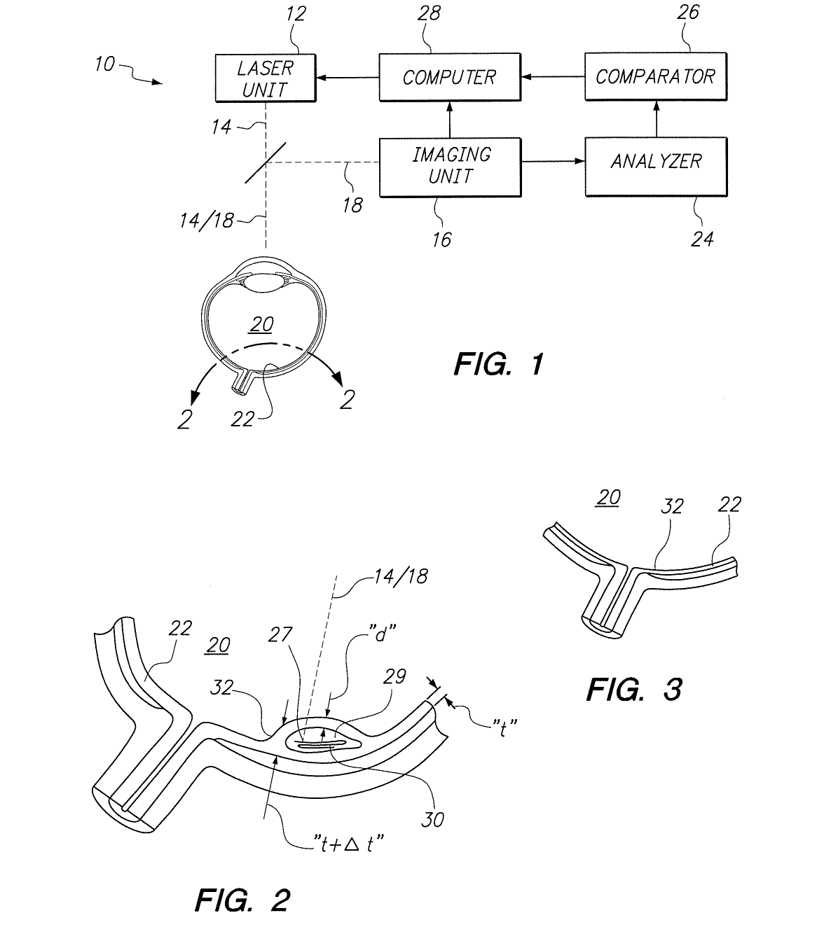

Referring initially to FIG. 1 a system for debulking retinal tissue in accordance with the present invention is shown and is generally designated 10. As shown, the system 10 includes a laser unit 12 for generating a laser beam 14. Preferably, the laser beam 14 is a pulsed femtosecond laser beam, wherein each pulse has a duration of less than about 500 femtoseconds. FIG. 1 also shows that the system 10 includes an imaging unit 16 for generating an imaging beam 18. Preferably, the imaging unit 16 is of a type well known in the pertinent art that employs Optical Coherence Tomography (OCT) techniques for the purpose of creating three dimensional images. In this case, imaging is done of the eye 20. More specifically, the imaging unit 16 is used to create an image of the retina 22 of the eye 20.

FIG. 1 also shows that the system 10 includes an analyzer 24 that is connected between the imaging unit 16 and a comparator 26. Also, the imaging unit 16 is connected directly to a computer 28. Operationally, within these connections, the analyzer 24 receives input from the imaging unit 16 for analysis and evaluation of the retina 22. The information derived from this analysis and evaluation by the analyzer 24 is then subsequently transferred to the comparator 26. On the other hand, with the image information that is passed directly from the imaging unit 16 to the computer 28, the computer/comparator 28/26 establishes input to the laser unit 12 for controlling movements of the focal point 27. Specifically, in response to this input control, the laser unit 12 directs the focal point 27 (see FIG. 2) of laser beam 14 for the Laser Induced Optical Breakdown (LIOB) of tissue in the retina 22 of the eye 20.

FIG. 2 indicates it can happen that tachyphylaxis will result in the formation of scar tissue 29 in the retina 22 of an eye 20. A consequence of this is that in the region of the retina 22 where the scar tissue 29 has formed, the normal thickness “t” of the retina 22 will be increased by a variable amount “Δt”. By employing the three dimensional capabilities of the imaging unit 16, this increase “Δt” can be detected by the imaging unit 16. Moreover, information based on the detection of “Δt” by the imaging unit 16 can be used by the analyzer 24 to measure, and determine, the exact location and extent of the scar tissue 29. Further, the build-up in volume of this scar tissue 29 in the retina 22 can be determined.

Using information regarding the location, extent and volume of the scar tissue 29 that is provided in images created by the imaging unit 16, a path 30 can be defined through the scar tissue 29. Importantly, when defining the path 30, it is necessary that the focal point 27 remain beyond the distance “d” from the surface 32 of retina 22. In most instances the distance “d” will be greater than approximately ten microns. Specifically, this is done to not adversely affect the surface 32. Then, using the imaging unit 16 to monitor movement of the focal point 27, laser unit 12 can be controlled to move the focal point 27 along the path 30 to debulk the scar tissue 29. As will be appreciated by the skilled artisan, this movement of the focal point 27 can be accomplished using closed loop feedback control techniques wherein deviations of the focal point 27 from the path 30 are minimized. The intended consequence of this is the removal of all scar tissue 29 from the retina 22, with a reestablishment of the retina surface 32 as substantially shown in FIG. 3.

While the particular OCT-Guided Femtosecond Laser to Measure a Retinal Surface for Use in Performing an Intra-Retinal Ablation as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for performing intra-retinal ablation to debulk scar tissue which comprises:
a laser unit for generating a laser beam, wherein the laser unit includes a means for focusing the laser beam to a focal point and a means for guiding the focal point along a predetermined path;
an imaging unit for creating a three dimensional image of a region of the retina of an eye, wherein the retina has a surface and the three dimensional image includes information of the thickness “t” and of variations “Δt” in the thickness of the retina in the region, and wherein the variations of thickness “Δt” are indicative of scar tissue in the retina;
an analyzer for evaluating the three dimensional retinal image to identify a location and an extent of the scar tissue inside the retina based on the variations of thickness “Δt”; and
a computer for controlling the laser unit in response to the identification of scar tissue in the image, to guide the focal point along a path, wherein the path is beyond a distance “d” in a posterior direction from the surface of the retina and the focal point is used to minimize “Δt” and debulk scar tissue inside the retina.

2. A system as recited in claim 1 wherein the focal point is a focal spot having a diameter of approximately ten microns.

3. A system as recited in claim 1 wherein the imaging unit is an Optical Coherence Tomography (OCT) device.

4. A system as recited in claim 1 wherein the distance “d” is greater than approximately ten microns.

5. A system as recited in claim 1 further comprising a comparator connected to the computer, and to the imaging unit, for monitoring the movement of the laser beam focal point, in real time, to measure a deviation of the focal point from the predetermined path in the three dimensional image, and for use of the measurement by the laser unit to minimize the deviation.

6. A system as recited in claim 1 wherein the scar tissue is the result of tachyphylaxis.

7. A system as recited in claim 1 wherein the intra-retinal ablation is accomplished by Laser Induced Optical Breakdown (LIOB).

8. A method for performing intra-retinal ablation which comprises the steps of:
generating a laser beam;
focusing the laser beam to a focal point;
creating a three dimensional image of a region of the retina of an eye wherein the three dimensional image includes information of the thickness “t” of the retina and of variations “Δt” in the thickness of the retina in the region, and wherein the variations of thickness “Δt” are indicative of scar tissue in the retina;
evaluating the three dimensional retinal image to identify a location and an extent of the scar tissue inside the retina; and
controlling a movement of the focal point along a path in response to the evaluating step to minimize “Δt” and debulk scar tissue inside the retina.

9. A method as recited in claim 8 wherein the creating step is accomplished using an Optical Coherence Tomography (OCT) device.

10. A method as recited in claim 8 wherein the retina has a surface and the method further comprises the steps of:
monitoring movement of the focal point in real time; and
maintaining the focal point inside the retina beyond a distance “d” from the surface of the retina.

11. A method as recited in claim 10 further comprising the steps of:
measuring a deviation of the focal point from the predetermined path in the three dimensional image; and
using a result of the measuring step in the controlling step to minimize the deviation.

12. A method as recited in claim 10 wherein the distance “d” is greater than approximately ten microns.

13. A method as recited in claim 8 wherein the scar tissue is the result of tachyphylaxis.

14. A method as recited in claim 8 wherein the intra-retinal ablation is accomplished by Laser Induced Optical Breakdown (LIOB).

15. A computer program product for moving the focal spot of a laser beam along a predetermined path through a selected volume in a transparent object, wherein the computer program product comprises program sections for respectively: creating a three dimensional image of the volume in the object wherein the three dimensional image includes information of the thickness “t” of the volume and of variations “Δt” in the thickness of the volume, and wherein the variations of thickness “Δt” are indicative of abnormal material in the volume; evaluating the three dimensional image to identify a location and an extent of the abnormal material inside the volume; and controlling a movement of the focal point along a predetermined path in the volume to debulk the abnormal material inside the volume.

16. A computer program product as recited in claim 15 wherein the volume has a surface and the computer program product further comprises program sections for respectively: monitoring movement of the focal point in real time; and maintaining the focal point inside the volume beyond a distance “d” from the surface of the volume.

17. A computer program product as recited in claim 15 wherein the computer program product further comprises program sections for respectively: measuring a deviation of the focal point from the predetermined path in the three dimensional image; and using control of the focal point to minimize the deviation.
